(19) United States
(12) Patent Application Publication
(10) Pub. No.: US 2026/0345055 A1
(43) Pub. Date: Nov. 5, 2026

(54) OPERATION OF IMPLANTABLE MEDICAL DEVICE SYSTEM TO DETERMINE ATRIAL FIBRILLATION RECURRENCE LIKELIHOOD

(71) Applicants: Medtronic, Inc., Minneapolis, MN (US); Politecnico di Milano, Milano (IT); Universidad de Zaragoza, Zaragoza (ES); Lunds Universitet, Lund (SE)

(72) Inventors: Francisco Javier Saiz Vivo, Maastricht (NL); Mirko de Melis, Maastricht (NL); Valentina D.A. Corino, Milano (IT); Luca Mainardi, Fagnano (IT); Alba Martin Yebra, Teruel (ES); Leif N.G. Sornmo, Lund (SE)

(21) Appl. No.: 19/487,220

(22) PCT Filed: May 28, 2024
(86) PCT No.: PCT/US2024/031258

§ 371 Date: Nov. 24, 2025

Related U.S. Application Data
(60) Provisional application No. 63/505,149, filed on May 31, 2023.

(51) Int. Cl. A61B 5/361; A61B 5/00; A61B 5/287

(52) U.S. Cl. CPC A61B 5/361; A61B 5/0002; A61B 5/287; A61B 5/4848; A61B 5/686; A61B 2560/0468

(57) ABSTRACT

In some examples a medical system comprising an implantable medical device and processing circuitry. The implantable medical device is configured to: sense a cardiac signal of a patient; detect a plurality of episodes of atrial fibrillation (AF) over a time period based on the cardiac signal; and store AF episode data for the plurality of episodes of AF detected over the time period. The processing circuitry configured to: determine, based on the AF episode data, a first parameter representing a prevalence over the time period of AF episodes and a second parameter representing a degree of temporal clustering of the AF episodes; determine a likelihood of recurrence of AF after an AF treatment procedure based on the first parameter and the second parameter; and transmit the likelihood to a computing device for presentation to a clinician.

This application claims the benefit of U.S. Provisional Patent Application Ser. No. 63/505,149, filed May 31, 2023, the entire content of which is incorporated herein by reference.

### TECHNICAL FIELD

The present technology is generally related to medical systems, and more particularly, to devices and methods for monitoring patient health using implantable medical devices.

### BACKGROUND

Modern medicine provides advanced surgical procedures and tools, as well as robust health monitoring technology for the entire spectrum of diseases and maladies. For example, cardiac ablation is a surgical procedure where a clinician uses heat or cold, or other energy, e.g., pulsed voltage fields, to ablate cardiac tissue, or to create small scars in the patient's heart. This scar tissue can block irregular electrical signals in the heart that can cause various arrhythmias.

One example of a cardiac ablation procedure is a pulmonary vein isolation (PVI) procedure, performed to treat atrial fibrillation (AF). With a PVI procedure, a clinician guides a set of instruments to the patient's heart through a vein or artery using a long tube or catheter. Among these instruments are tools to deliver energy to ablate the location where the pulmonary veins connect with the left atrium.

### SUMMARY

Some patients are more likely to respond to or benefit from PVI or other catheter ablation procedures to treat AF than other patients. Furthermore, such procedures are associated with significant economic cost and patient burden. Consequently, it would be advantageous to determine, prior to a procedure, whether the patient is likely to benefit from the procedure.

According to the techniques of this disclosure, an insertable cardiac monitor (ICM) or other implantable medical device (IMD) detects episodes of AF of a patient during a period of time preceding a decision of whether to undertake a procedure, e.g., PVI, to treat the patient's AF. Based on AF episode data for the detected AF episodes stored by the IMD, processing circuitry of a medical system including the IMD may determine a likelihood of recurrence of AF after the AF treatment procedure. The processing circuitry determines the likelihood of recurrence of AF based on a unique combination of parameters, e.g., a first parameter representing a prevalence over the time of AF episodes and a second parameter representing a degree of temporal clustering of the AF episodes. Such a combination of parameters provides a technical advantage of improved accuracy for a prediction of whether a patient will benefit from an AF treatment procedure, e.g., relative to conventional techniques that rely on a single metric of AF experienced by the patient, e.g., AF burden/prevalence.

The techniques of this disclosure may be implemented by systems including one or more IMDs that can autonomously and continuously detect AF episodes and store AF episode data while the IMD is implanted in a patient over months or years and perform numerous operations per second on the data to enable the systems herein to determine a likelihood of AF recurrence for the patient. Using techniques of this disclosure with an IMD may be advantageous when a physician cannot be continuously present with the patient to evaluate the physiological parameters and/or where performing the operations on the data described herein could not practically be performed in the mind of a physician.

In some examples, the techniques and systems of this disclosure may use one or more models, e.g., probability and/or machine learning models, to more accurately determine the likelihood of AF recurrence based on the AF episode data collected by the IMD. In some examples, the model is trained with a set of training instances, where one or more of the training instances comprise data that indicate relationships between AF episode data and likelihood values. Because the model is configured based on potentially thousands or millions of training instances, the model may reduce the amount of error in recurrence prediction performed by the system. Reducing errors using the techniques of this disclosure may provide one or more technical and clinical advantages, such as increasing the likelihood that AF treatment procedures are provided to patients that will benefit from the procedures.

In one example, a medical system comprise an implantable medical device configured to: sense a cardiac signal of a patient; detect a plurality of episodes of atrial fibrillation (AF) over a time period based on the cardiac signal; and store AF episode data for the plurality of episodes of AF detected over the time period. The medical system further comprises processing circuitry configured to: determine, based on the AF episode data, a first parameter representing a prevalence over the time of AF episodes and a second parameter representing a degree of temporal clustering of the AF episodes; determine a likelihood of recurrence of AF after an AF treatment procedure based on the first parameter and the second parameter; and transmit the likelihood to a computing device for presentation to a clinician.

In another example, a method of operating a medical system comprising an implantable medical device configured to sense a cardiac signal of a patient and detect a plurality of episodes of atrial fibrillation (AF) over a time period based on the cardiac signal, the method comprises: determining, by processing circuitry of the medical system and based on AF episode data stored by the implantable medical device for the plurality of episodes of AF detected over the time period, a first parameter representing a prevalence over the time of AF episodes and a second parameter representing a degree of temporal clustering of the AF episodes; determining, by the processing circuitry, a likelihood of recurrence of AF after an AF treatment procedure based on the first parameter and the second parameter; and transmitting, by the processing circuitry, the likelihood to a computing device for presentation to a clinician.

In another example, a non-transitory computer-readable storage medium comprising program instructions that, when executed by processing circuitry of a medical system comprising an implantable medical device configured to sense a cardiac signal of a patient and detect a plurality of episodes of atrial fibrillation (AF) over a time period based on the cardiac signal, cause the processing circuitry to: determine, based on AF episode data stored by the implantable medical device for the plurality of episodes of AF detected over the time period, a first parameter representing a prevalence over the time of AF episodes and a second parameter representing a degree of temporal clustering of the AF episodes; determine a likelihood of recurrence of AF after an AF treatment procedure based on the first parameter and the second parameter; and transmit the likelihood to a computing device for presentation to a clinician.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF THE DRAWINGS

FIG. 1 is a block diagram illustrating an example medical system including an IMD in conjunction with a patient, according to some aspects of this disclosure.

FIG. 2A is a perspective drawing illustrating an example IMD.

FIG. 2B is a perspective drawing illustrating another example IMD.

FIG. 3 is a block diagram illustrating an example configuration of an IMD.

FIG. 4 is a block diagram illustrating an example configuration of a computing device that operates in accordance with one or more techniques of the present disclosure.

FIG. 5 is a block diagram illustrating an example configuration of a health monitoring system that operates in accordance with one or more techniques of the present disclosure.

FIG. 6 is a conceptual diagram illustrating an example machine learning model configured to determine a likelihood of recurrence of AF.

FIG. 7 is a conceptual diagram illustrating an example training process for an artificial intelligence model, in accordance with examples of the current disclosure.

FIG. 8 is a set of charts showing a heart rhythm condition over time, heart rhythm condition transition times, a first conditional intensity function, and a second conditional intensity function according to some aspects of this disclosure.

FIGS. 9A-9D are charts showing varying degrees of prevalence over time of AF episodes and clustering of AF episodes according to some aspects of this disclosure.

FIG. 10 is a flow chart illustrating an example of a process for determining a likelihood of recurrence of AF according to some aspects of this disclosure.

### DETAILED DESCRIPTION

AF is characterized by abnormal or irregular beating of the atrial chambers of a patient's heart. With paroxysmal AF (PAF), patients can suffer from episodes of AF intermittently, where the episodes terminate spontaneously (e.g., the heart returns to a normal sinus rhythm (SR)). AF can be a progressive disease, with AF episodes sometimes leading to sustained forms of AF.

One surgical treatment for AF is a pulmonary vein isolation (PVI) procedure. With PVI, a clinician ablates (e.g., by freezing) tissue in the left atrium to electrically isolate the left atrium from the pulmonary veins, where the abnormal electrical activity causing the AF may originate. However, a PVI procedure may not be effective in the long term for all patients. That is, for many patients, there remains a risk of recurrence of AF after a PVI procedure. In some aspects, the present disclosure provides for the determination of a pre-ablation risk assessment for a patient based on AF episode patterns. That is, by analyzing heart rhythm condition classification data over time, the likelihood or risk of post-ablation recurrence of AF can be characterized.

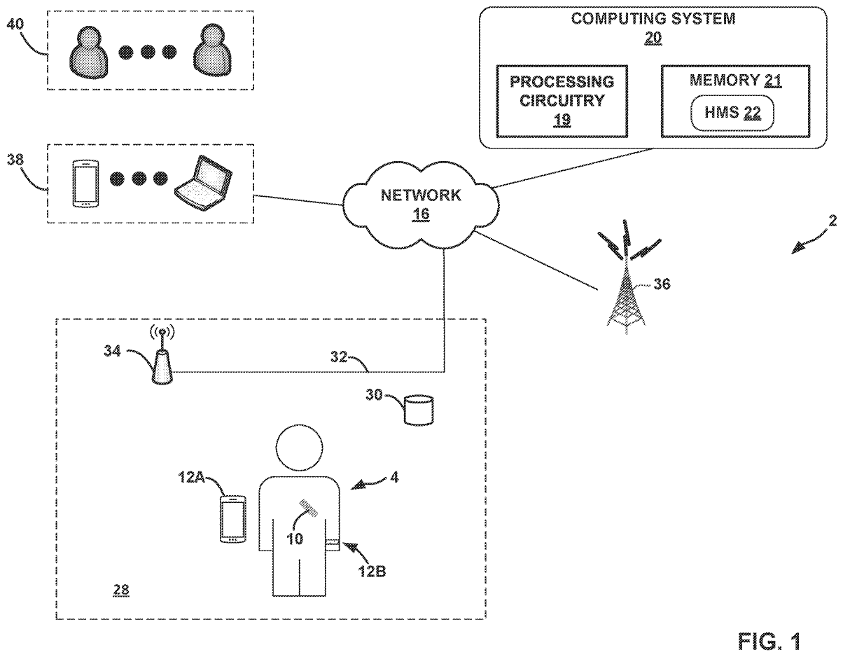

FIG. 1 is a block diagram illustrating an example medical system 2 including an IMD 10 in conjunction with a patient 4, according to some aspects of this disclosure. IMD 10 is configured for continuous, long-term monitoring of the heart of patient 4. For example, IMD 10 is configured to sense an electrocardiogram (ECG) or other cardiac signal, detected episodes of AF based on the cardiac signal, and store AF episode data describing parameters or metrics of the detected AF episodes.

As examples, IMD 10 may be a pacemaker or implantable cardioverter-defibrillator, which may be coupled to intravascular or extravascular leads, or a pacemaker with a housing configured for implantation within the heart, which may be leadless. Some IMDs do not provide therapy, such as implantable patient monitors. IMD 10 may be such an IMD, e.g., the Reveal LINQ™ or LINQ II™ Insertable Cardiac Monitor (ICM), available from Medtronic, Inc., of Minneapolis, Minnesota, which may be inserted subcutaneously. Such IMDs may facilitate relatively longer-term and continuous monitoring of patients during normal daily activities, and may periodically transmit collected data to a remote patient monitoring system, such as the Medtronic Carelink™ Network.

IMD 10 may determine values of patient parameters or metrics, e.g., based on physiological signals sensed by the IMD or responsive therapies delivered by the IMD. The patient parameters may include, as examples, fluid level, heart rate, respiration rate, patient activity, temperature, heart sounds, oxygenation, and R-wave morphological characteristics. Other example patient metrics include coughing, speech, posture, tissue perfusion, hematocrit, thoracic impedance, subcutaneous impedance, intracardiac impedance, heart rate variability (HRV), weight, blood pressure, sleep apnea burden (which may be derived from respiration rate), ischemia burden, sleep duration, sleep quality, PVC burden, the occurrence, frequency or duration cardiac arrhythmias or other events, such as AF, and sensed cardiac intervals (e.g., Q-T intervals). Another example patient metric is the ventricular rate during AF. The concentration or levels of various substances, such as blood glucose, hematocrit, troponin and/or brain natriuretic peptide (BNP) levels, within the patient may also be used as one or more patient metrics.

In addition to IMD 10, system 2 includes one or more patient computing devices, e.g., patient computing devices 12A and 12B (collectively, “patient computing devices 12”). Patient computing device(s) 12 are configured for wireless communication with IMD 10. Computing device(s) 12 retrieve parameter data, e.g., AF episode data, and other data from IMD 10. In some examples, computing device(s) 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing device(s) 12 may communicate with IMD 10 and each other according to the Bluetooth® or Bluetooth® Low Energy (BLE) protocols, as examples. In some examples, only one of computing device(s) 12, e.g., computing device 12A, is configured for communication with IMD 10, e.g., due to execution of software (e.g., part of a health monitoring application as described herein) enabling communication and interaction with an IMD.

In some examples, computing device(s) 12, e.g., wearable computing device 12B in the example illustrated by FIG. 1, may include electrodes and other sensors to sense physiological signals of patient 4, and may collect and store parameter data based on such signals. Computing device 12B may be incorporated into the apparel of patient 4, such as within clothing, shoes, eyeglasses, a watch or wristband, a hat, etc. In some examples, computing device 12B is a smartwatch or other accessory or peripheral for a smartphone computing device 12A.

One or more of computing device(s) 12 may be configured to communicate with a variety of other devices or systems via a network 16. For example, one or more of computing device(s) 12 may be configured to communicate with one or more computing systems, e.g., computing system 20, via network 16. Computing system 20 may be managed by a manufacturer of IMD 10 to, for example, provide cloud storage and analysis of collected data, maintenance and software services, or other networked functionality for their respective devices and users thereof. Computing system 20 may comprise, or may be implemented by, the Medtronic Carelink™ Network, in some examples.

As illustrated in FIG. 1, computing system 20 may include processing circuitry 19 and memory 22. Processing circuitry 19 may include fixed function circuitry and/or programmable processing circuitry. Processing circuitry 19 may include any one or more of a microprocessor, a controller, a digital signal processor (DSP), graphics processing unit (GPU), tensor processing unit (TPU), an application specific integrated circuit (ASIC), a field-programmable gate array (FPGA), or equivalent discrete or analog logic circuitry. In some examples, processing circuitry 19 may include multiple components, such as any combination of one or more microprocessors, one or more controllers, one or more DSPs, GPUs, TPUs, one or more ASICs, or one or more FPGAs, as well as other discrete or integrated logic circuitry, which may be physically located in one or more devices in one or more physical locations. Computing system 20 may be configured as a cloud computing system.

Processing circuitry 19 may be capable of processing instructions stored in memory 21. In some examples, memory 21 includes a computer-readable medium that includes instructions that, when executed by processing circuitry 19, cause computing system 20 and processing circuitry 19 to perform various functions attributed to them herein. In the example illustrated by FIG. 1, computing system 20 implements a health monitoring system (HMS) 22. As will be described in greater detail below, HMS 22 may help determine a likelihood of recurrence of AF after an AF treatment procedure. Memory 21 may include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), dynamic random-access memory (DRAM), flash memory, or any other digital media.

Computing device(s) 12 may transmit data, including data retrieved from IMD 10, to computing system 20 via network 16. The data may include sensed data, e.g., values of physiological parameters measured by IMD 10 and, in some cases one or more of computing device(s) 12, and other physiological signals or data recorded by IMD 10 and/or computing device(s) 12. The data transmitted from computing device(s) 12 to computing system 20 may include AF episode data.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, IMD 10 may be configured to generate diagnostic information of patient 4, such as AF episode data. In some examples, IMD 10 may be configured to transmit such data to wireless access point 34 and/or computing device(s) 12. Wireless access points 34 and/or computing device(s) 12 may then communicate the retrieved data to computing systems 20 via network 16.

In some cases, computing system 20 may be configured to provide a secure storage site for data that has been collected from IMD 10 and/or computing device(s) 12. In some instances, computing system 20 may include a database that stores medical- and health-related data. For example, computing system 20 may include a cloud server or other remote server that stores data collected from IMDs 10 and/or computing device(s) 12. In some cases, computing system 20 may assemble data in web pages or other documents for viewing by trained professionals, such as clinicians 40, via clinician computing devices 38. One or more aspects of the example system described with reference to FIG. 1 may be implemented with general network technology and functionality, which may be similar to that provided by the Medtronic CareLink® Network.

In some examples, one or more of clinician computing devices 38 may be a tablet or other smart device located with a clinician, by which the clinician may program, receive alerts from, and/or interrogate IMD 10. For example, the clinician may access data collected by IMD 10 through a clinician computing device 38, such as when patient 4 is in between clinician visits, to check on a status of a medical condition. For example, computing system 20 may transmit data indicating a likelihood of AF recurrence (e.g., determined by computing system 20, computing device 12, or other devices described herein) to clinician(s) 40 via computing device(s) 38.

As will be described herein, processing circuitry of one or more devices of system 2 (e.g., of computing device 12 and/or computing system 20 implementing HMS 22) may be configured to determine, prior to an AF treatment procedure, a likelihood of recurrence of AF after the procedure based on AF episode data generated by IMD 10. To determine the likelihood of recurrence, the processing circuitry may determine values of a first parameter and a second parameter based on the AF episode data. The first parameter may represent a prevalence over the time of AF episodes and the second parameter representing a degree of temporal clustering of the AF episodes.

In the example of FIG. 1, environment 28 includes one or more Internet of Things (IOT) devices, such as IoT device 30. IoT device 30 may include, as examples, so called “smart” speakers, cameras, televisions, lights, locks, thermostats, appliances, actuators, controllers, or any other smart home (or building) devices. In the example of FIG. 1, IoT device 30 is a smart speaker and/or controller, which may include a display. In some examples, IoT device 30 includes cameras or other sensors may activate those sensors to collect data regarding patient 4, e.g., for evaluation of the condition of patient 4.

Computing device(s) 12 may be configured to wirelessly communicate with IoT device 30 to cause IoT device 30 to take the actions described herein. In some examples, HMS 22 communicates with IoT device 30 via network 16 to cause IoT device 30 to take the actions described herein. In some examples, IMD 10 is configured to communicate wirelessly with IT device 30, e.g., to communicate data to computing system 20 via network 16. In some examples, IoT device 30 may be configured to provide some or all of the functionality ascribed to computing device(s) 12 herein.

Environment 28 includes computing facilities, e.g., a local network 32, by which computing device(s) 12, IoT device 30, and other devices within environment 28 may communicate via network 16, e.g., with HMS 22. For example, environment 28 may be configured with wireless technology, such as IEEE 802.11 wireless networks, IEEE 802.15 ZigBee networks, an ultra-wideband protocol, near-field communication, or the like. Environment 28 may include one or more wireless access points, e.g., wireless access point 34 that provides support for wireless communications throughout environment 28. Additionally or alternatively, e.g., when local network is unavailable, computing device(s) 12, IoT devices 30, and other devices within environment 28 may be configured to communicate with network 16, e.g., with HMS 22, via a cellular base station 36 and a cellular network.

In some examples, computing device(s) 12 and/or computing system 20 may implement one or more algorithms to determine likelihood of AF recurrence based on data received from IMD 10. In some examples, computing device(s) 12 and/or computing system 20 may have greater processing capacity than IMD 10, enabling more complex analysis of the data. In some examples, the computing device(s) 12 and/or HMS 22 may apply the data to a probability model, machine learning model or other artificial intelligence developed algorithm, e.g., to determine the likelihood of recurrence or the parameters described herein.

Although described in the context of examples in which IMD 10 that senses patient cardiac activity may comprise an ICM, example systems including one or more implantable, wearable, or external devices of any type configured to sense physiological parameters of a patient may be configured to implement the techniques of this disclosure.

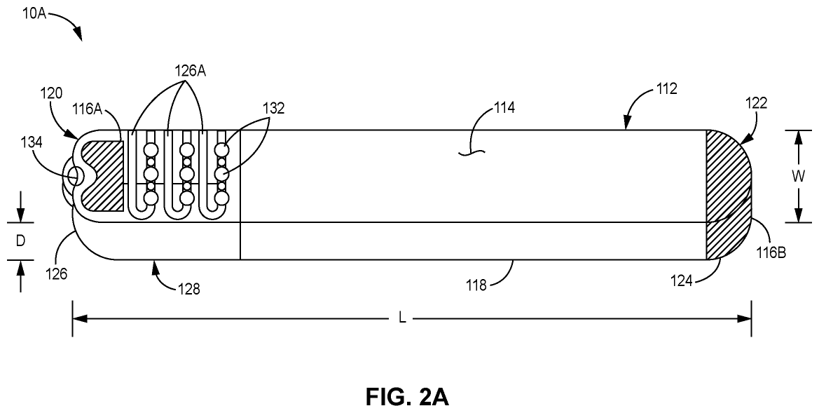

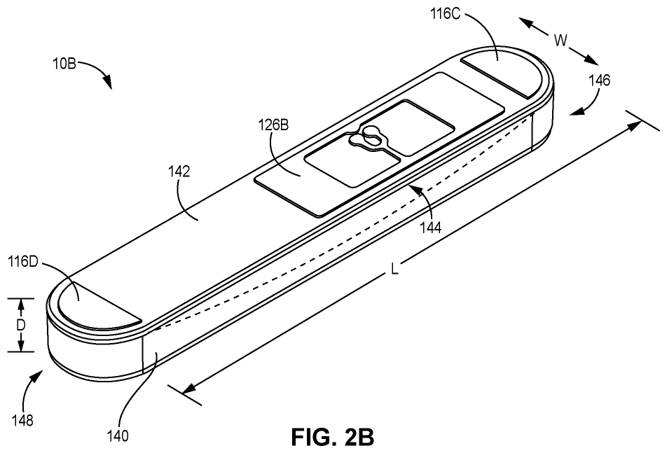

FIG. 2A is a perspective drawing illustrating an IMD 10A, which may be an example configuration of IMD 10 of FIG. 1 as an ICM. In the example shown in FIG. 2A, IMD 10A may be embodied as a monitoring device having housing 112, proximal electrode 116A and distal electrode 16B. Housing 112 may further comprise first major surface 114, second major surface 118, proximal end 120, and distal end 122. Housing 112 encloses electronic circuitry located inside the IMD 10A and protects the circuitry contained therein from body fluids. Housing 112 may be hermetically sealed and configured for subcutaneous implantation. Electrical feedthroughs provide electrical connection of electrodes 116A and 116B.

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion. For example, the device shown in FIG. 2A includes radial asymmetries (notably, the rectangular shape) along the longitudinal axis that maintains the device in the proper orientation following insertion. For example, the spacing between proximal electrode 116A and distal electrode 116B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 114 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 2A, once inserted within the patient, the first major surface 114 faces outward, toward the skin of the patient while the second major surface 118 is located opposite the first major surface 114. In addition, in the example shown in FIG. 2A, proximal end 120 and distal end 122 are rounded to reduce discomfort and irritation to surrounding tissue once inserted under the skin of the patient. IMD 10A, including instrument and method for inserting IMD 10A is described, for example, in U.S. Patent Publication No. 2014/0276928, incorporated herein by reference in its entirety.

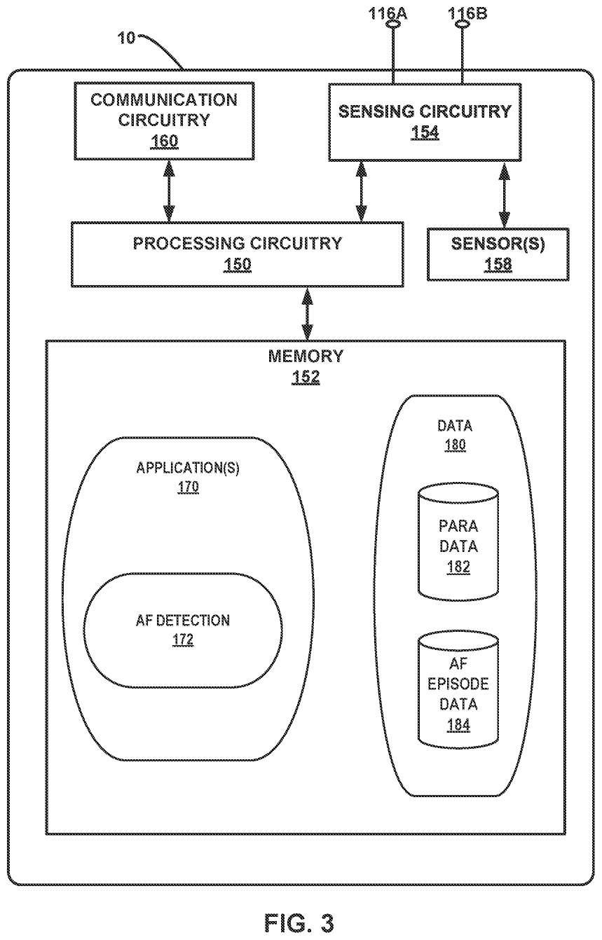

Proximal electrode 116A is at or proximate to proximal end 120, and distal electrode 116B is at or proximate to distal end 122. Proximal electrode 116A and distal electrode 116B are used to sense cardiac signals, e.g., ECG signals, and measure interstitial impedance thoracically outside the ribcage, which may be sub-muscularly or subcutaneously. ECG signals and impedance measurements may be stored in a memory of IMD 10A, and data may be transmitted via integrated antenna 126A to another device, which may be another implantable device or an external device, such as computing device 12. In some example, electrodes 116A and 116B may additionally or alternatively be used for sensing any bio-potential signal of interest, which may be, for example, an electrogram (EGM), electroencephalogram (EEG), electromyogram (EMG), or a nerve signal, from any implanted location. Housing 112 may house the circuitry of IMD 10 illustrated in FIG. 3.

In the example shown in FIG. 2A, proximal electrode 116A is at or in close proximity to the proximal end 120 and distal electrode 116B is at or in close proximity to distal end 122. In this example, distal electrode 116B is not limited to a flattened, outward facing surface, but may extend from first major surface 114 around rounded edges 124 and/or end surface 126 and onto the second major surface 118 so that the electrode 116B has a three-dimensional curved configuration. In some examples, electrode 116B is an uninsulated portion of a metallic, e.g., titanium, part of housing 112.

In the example shown in FIG. 2A, proximal electrode 116A is located on first major surface 114 and is substantially flat, and outward facing. However, in other examples proximal electrode 116A may utilize the three dimensional curved configuration of distal electrode 116B, providing a three dimensional proximal electrode (not shown in this example). Similarly, in other examples distal electrode 116B may utilize a substantially flat, outward facing electrode located on first major surface 114 similar to that shown with respect to proximal electrode 116A.

The various electrode configurations allow for configurations in which proximal electrode 116A and distal electrode 116B are located on both first major surface 114 and second major surface 118. In other configurations, such as that shown in FIG. 2A, only one of proximal electrode 116A and distal electrode 116B is located on both major surfaces 114 and 118, and in still other configurations both proximal electrode 116A and distal electrode 116B are located on one of the first major surface 114 or the second major surface 118 (e.g., proximal electrode 116A located on first major surface 114 while distal electrode 116B is located on second major surface 118). In another example, IMD 10A may include electrodes on both major surface 114 and 118 at or near the proximal and distal ends of the device, such that a total of four electrodes are included on IMD 10A. Electrodes 116A and 116B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 2A, proximal end 120 includes a header assembly 128 that includes one or more of proximal electrode 116A, integrated antenna 126A, anti-migration projections 132, and/or suture hole 134. Integrated antenna 126A is located on the same major surface (i.e., first major surface 114) as proximal electrode 116A and is also included as part of header assembly 128. Integrated antenna 126A allows IMD 10A to transmit and/or receive data. In other examples, integrated antenna 126A may be formed on the opposite major surface as proximal electrode 116A, or may be incorporated within the housing 112 of IMD 10A. In the example shown in FIG. 2A, anti-migration projections 132 are located adjacent to integrated antenna 126A and protrude away from first major surface 114 to prevent longitudinal movement of the device. In the example shown in FIG. 2A, anti-migration projections 132 include a plurality (e.g., nine) small bumps or protrusions extending away from first major surface 114. As discussed above, in other examples anti-migration projections 132 may be located on the opposite major surface as proximal electrode 116A and/or integrated antenna 126A. In addition, in the example shown in FIG. 2A, header assembly 128 includes suture hole 134, which provides another means of securing IMD 10A to the patient to prevent movement following insertion. In the example shown, suture hole 134 is located adjacent to proximal electrode 116A. In one example, header assembly 128 is a molded header assembly made from a polymeric or plastic material, which may be integrated or separable from the main portion of IMD 10A.

FIG. 2B is a perspective drawing illustrating another IMD 10B, which may be another example configuration of IMD 10 from FIG. 1 as an ICM. IMD 10B of FIG. 2B may be configured substantially similarly to IMD 10A of FIG. 2A, with differences between them discussed herein.

IMD 10B may include a leadless, subcutaneously-implantable monitoring device, e.g. an ICM. IMD 10B includes housing having a base 140 and an insulative cover 142. Proximal electrode 116C and distal electrode 116D may be formed or placed on an outer surface of cover 142. Various circuitries and components of IMD 10B, e.g., described with respect to FIG. 3, may be formed or placed on an inner surface of cover 142, or within base 140. In some examples, a battery or other power source of IMD 10B may be included within base 140. In the illustrated example, antenna 126B is formed or placed on the outer surface of cover 142, but may be formed or placed on the inner surface in some examples. In some examples, insulative cover 142 may be positioned over an open base 140 such that base 140 and cover 142 enclose the circuitries and other components and protect them from fluids such as body fluids. The housing including base 140 and insulative cover 142 may be hermetically sealed and configured for subcutaneous implantation.

Circuitries and components may be formed on the inner side of insulative cover 142, such as by using flip-chip technology. Insulative cover 142 may be flipped onto a base 140. When flipped and placed onto base 140, the components of IMD 10B formed on the inner side of insulative cover 142 may be positioned in a gap 144 defined by base 140. Electrodes 116C and 116D and antenna 126B may be electrically connected to circuitry formed on the inner side of insulative cover 142 through one or more vias (not shown) formed through insulative cover 942. Insulative cover 142 may be formed of sapphire (i.e., corundum), glass, parylene, and/or any other suitable insulating material. Base 140 may be formed from titanium or any other suitable material (e.g., a biocompatible material). Electrodes 116C and 116D may be formed from any of stainless steel, titanium, platinum, iridium, or alloys thereof. In addition, electrodes 116C and 116D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between proximal electrode 116C and distal electrode 116D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within the patient, outer surface of cover 142 faces outward, toward the skin of the patient. In addition, as shown in FIG. 2B, proximal end 146 and distal end 148 are rounded to reduce discomfort and irritation to surrounding tissue once inserted under the skin of the patient. In addition, edges of IMD 10B may be rounded.

FIG. 3 is a block diagram illustrating an example configuration of IMD 10 of FIG. 1. As shown in FIG. 3, IMD 10 includes processing circuitry 150, memory 152, sensing circuitry 154 coupled to electrodes 116A and 116B (hereinafter, “electrodes 116”) and one or more sensor(s) 158, and communication circuitry 160.

Processing circuitry 150 may include fixed function circuitry and/or programmable processing circuitry. Processing circuitry 150 may include any one or more of a microprocessor, a controller, a GPU, a TPU, a DSP, an ASIC, a FPGA, or equivalent discrete or analog logic circuitry. In some examples, processing circuitry 150 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 150 herein may be embodied as software, firmware, hardware, or any combination thereof. In some examples, memory 152 includes computer-readable instructions that, when executed by processing circuitry 150, cause IMD 10 and processing circuitry 150 to perform various functions attributed herein to IMD 10 and processing circuitry 150. Memory 152 may include any volatile, non-volatile, magnetic, optical, or electrical media, such as a RAM, ROM, NVRAM, EEPROM, flash memory, or any other digital media.

Sensing circuitry 154 may sense an ECG and measure impedance, e.g., of tissue proximate to IMD 10, via electrodes 116. The measured impedance may vary based on respiration, cardiac pulse or flow, and a degree of perfusion or edema. Processing circuitry 150 may determine patient metrics relating to respiration, fluid retention, cardiac pulse or flow, perfusion, and/or edema based on the measured impedance. In some examples, processing circuitry 150 may identify features of the sensed ECG, such as heart rate, heart rate variability, T-wave alternans, intra-beat intervals (e.g., QT intervals), and/or ECG morphologic features, to detect an episode of cardiac arrhythmia of patient 4.

In some examples, IMD 10 includes one or more sensors 158, such as one or more accelerometers, gyroscopes, microphones, optical sensors, temperature sensors, pressure sensors, and/or chemical sensors. In some examples, sensing circuitry 152 may include one or more filters and amplifiers for filtering and amplifying signals received from one or more of electrodes 116 and/or sensors 158. In some examples, sensing circuitry 154 and/or processing circuitry 150 may include a rectifier, filter and/or amplifier, a sense amplifier, comparator, and/or analog-to-digital converter. Processing circuitry 150 may determine physiological parameter data 182, e.g., values of physiological parameters of patient 4, based on signals from sensors 158, which may be stored as data 180 in memory 152. Patient parameters determined from signals from sensors 158 may include intravascular fluid level, interstitial fluid level, oxygen saturation, glucose level, stress hormone level, heart sounds, body motion, activity intensity, sleep duration, sleep quality, body posture, or blood pressure.

In addition to data 180, memory 152 may store applications 170 executable by processing circuitry 150. Applications 170 may include an AF detection application 172. Processing circuitry 150 may execute AF detection application 172 to detect episodes of AF of patient 4 based on an ECG signal sensed by sensing circuitry 154 via electrodes 116. In some examples, processing circuitry 150 may detect AF episodes based on other cardiac signals, e.g., impedance, optical, pressure, accelerometer, or other signals that vary based on mechanical activity of the heart. In some examples, AF detection application 172 detects AF based on rate and regularity (e.g., variability or dispersion) of intervals between consecutive depolarizations or contractions of the heart, e.g., R-R intervals. In one example, AF detection application 172 may be based on an R-R interval pattern-based algorithm and a P-wave evidence score, which reduces false positive AF detections and leverages the evidence of a single P-wave between two R waves using morphologic processing of the ECG signal.

Processing circuitry 150 may store data 180 of various metrics of one or more AF episodes detected by the processing circuitry as AF episode data 184. AF episode data 184 may include start time, stop time, and/or duration for each episode. AF episode data 184 may include heart rate information, R-R interval information, ECG morphological information, or other parameter data 182 during AF episodes. AF episode data 184 may also include AF burden information for the one or detected AF episodes. AF burden information may include total durations of AF per period, e.g., total AF minutes per day.

Processing circuitry 150 may communicate AF episode data 184 and other parameter data 182 to one or more other computing devices, e.g., computing device(s) 12 and/or computing system 20, using communication circuitry 160. Communication circuitry 160 may include any suitable hardware, firmware, software or any combination thereof for wirelessly communicating with another device. Communication circuitry 162 may be configured to transmit and/or receive signals via inductive coupling, electromagnetic coupling, Near Field Communication (NFC), Radio Frequency (RF) communication, Bluetooth®, WiFi, or other proprietary or non-proprietary wireless communication schemes.

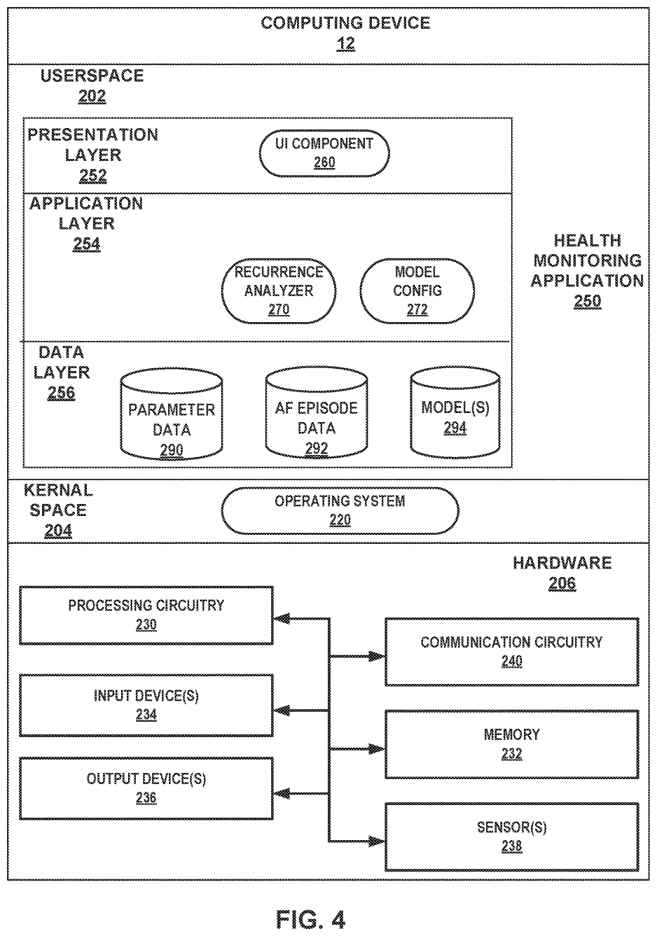

FIG. 4 is a block diagram illustrating an example configuration of a computing device 12, which may correspond to either (or both operating in coordination) of computing devices 12A and 12B. In some examples, computing device 12 takes the form of a smartphone, a laptop, a tablet computer, a personal digital assistant (PDA), a smartwatch or other wearable computing device. In some examples, IoT devices 30 and/or computing devices 38 and 42 may be configured similarly to the configuration of computing device 12 illustrated in FIG. 4.

As shown in the example of FIG. 4, computing device 12 may be logically divided into user space 202, kernel space 204, and hardware 206. Hardware 206 may include one or more hardware components that provide an operating environment for components executing in user space 202 and kernel space 204. User space 202 and kernel space 204 may represent different sections or segmentations of memory, where kernel space 204 provides higher privileges to processes and threads than user space 202. For instance, kernel space 204 may include operating system 220, which operates with higher privileges than components executing in user space 202.

As shown in FIG. 4, hardware 206 includes processing circuitry 230, memory 232, one or more input devices 234, one or more output devices 236, one or more sensors 238, and communication circuitry 240. Although shown in FIG. 4 as a stand-alone device for purposes of example, computing device 12 may be any component or system that includes processing circuitry or other suitable computing environment for executing software instructions and, for example, need not necessarily include one or more elements shown in FIG. 4.

Processing circuitry 230 is configured to implement functionality and/or process instructions for execution within computing device 12. For example, processing circuitry 230 may be configured to receive and process instructions stored in memory 232 that provide functionality of components included in kernel space 204 and user space 202 to perform one or more operations in accordance with techniques of this disclosure. Examples of processing circuitry 230 may include, any one or more microprocessors, controllers, GPUs, TPUs, DSPs, ASICs, FPGAs, or equivalent discrete or integrated logic circuitry.

Memory 232 may be configured to store information within computing device 12, for processing during operation of computing device 12. Memory 232, in some examples, is described as a computer-readable storage medium. In some examples, memory 232 includes a temporary memory or a volatile memory. Examples of volatile memories include RAM, DRAM, SRAM, and other forms of volatile memories known in the art. Memory 232, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 232 includes cloud-associated storage.

One or more input devices 234 of computing device 12 may receive input, e.g., from patient 4, clinician 40, or another user. Examples of input are tactile, audio, kinetic, and optical input. Input devices 234 may include, as examples, a mouse, keyboard, voice responsive system, camera, buttons, control pad, microphone, presence-sensitive or touch-sensitive component (e.g., screen), or any other device for detecting input from a user or a machine.

One or more output devices 236 of computing device 12 may generate output, e.g., to patient 4 or another user. Examples of output are tactile, haptic, audio, and visual output. Output devices 236 of computing device 12 may include a presence-sensitive screen, sound card, video graphics adapter card, speaker, cathode ray tube monitor, liquid crystal display (LCD), light emitting diodes (LEDs), or any type of device for generating tactile, audio, and/or visual output.

One or more sensors 238 of computing device 12 may sense physiological parameters or signals of patient 4. Sensor(s) 238 may include electrodes, accelerometers (e.g., 3-axis accelerometers), an optical sensor, impedance sensors, temperature sensors, pressure sensors, heart sound sensors (e.g., microphones or accelerometers), and other sensors, and sensing circuitry (e.g., including an ADC), similar to those described above with respect to IMDs 10 and FIG. 3.

Communication circuitry 240 of computing device 12 may communicate with other devices by transmitting and receiving data. Communication circuitry 240 may receive data from IMD 10, such as patients metrics and/or higher resolution diagnostic information, from communication circuitry in IMD 10. Communication circuitry 240 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 4, health monitoring application 250 executes in user space 202 of computing device 12. Health monitoring application 250 may be logically divided into presentation layer 252, application layer 254, and data layer 256. Presentation layer 252 may include a user interface (UI) component 260, which generates and renders user interfaces of health monitoring application 250.

Data layer 256 may include parameter data 290 and AF episode data 292, which may be received from IMD 10 via communication circuitry 240, and stored in memory 232 by processing circuitry 230. Application layer 254 may include, but is not limited to, recurrence analyzer 270, and model configuration application 272. Recurrence analyzer 270 may determine a likelihood of AF recurrence after an AF treatment procedure based on AF episode data 292, and in some cases other parameter data 290, generated by IMD 10 based on AF episodes detected during an observation time period prior to deciding whether to perform the procedure for patient 4. Recurrence analyzer 270 may determine the likelihood of recurrence based on application of the data as inputs one or more model(s) 294, which may include one or more probability models, machine learning models, algorithms, decision trees, and/or thresholds. In examples in which models 294 include one or more machine learning models, recurrence analyzer 270 may apply feature vectors derived from the data to the model(s).

Model configuration component 272 may be configured to modify models 294 based on feedback indicating whether determinations were accurate or updated parameters received, e.g., from HMS 22. In some examples, model configuration component 272 may utilize the data sets from patient 4 for supervised machine learning to further train models included as part of models 294. Model configuration component 272, or another component executed by processing circuitry of system 2, may select a configuration of models 294 based on etiological data for patient. In some examples, different model(s) 294 tailored to different cohorts of patients may be available for selection for patient 4 based on such etiological data.

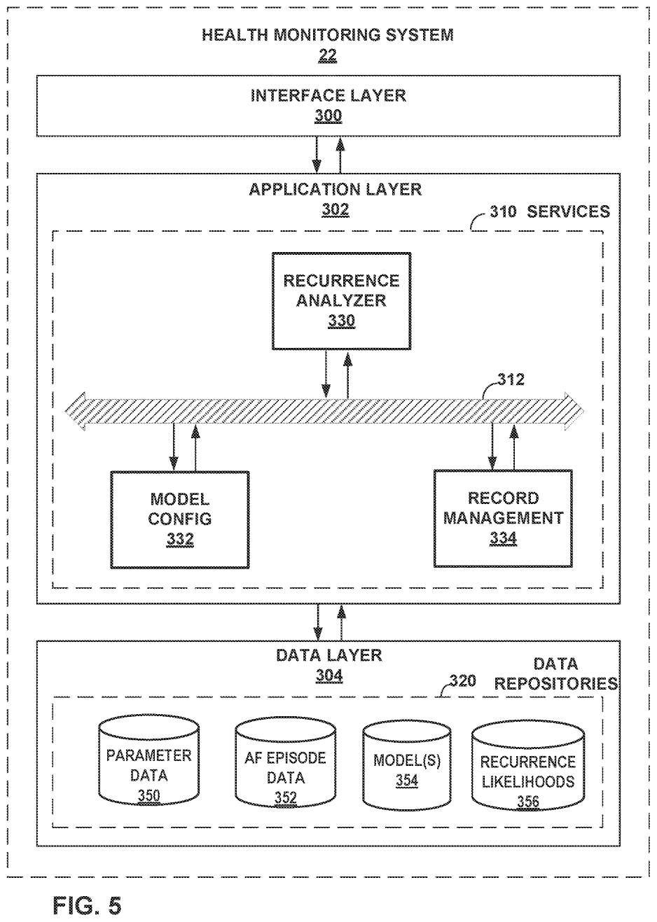

FIG. 5 is a block diagram illustrating an operating perspective of HMS 22. HMS 22 may be implemented in a computing system 20, which may include hardware components such as processing circuitry 19, memory 21, and communication circuitry, embodied in one or more physical devices. FIG. 5 provides an operating perspective of HMS 22 when hosted as a cloud-based platform. In the example of FIG. 5, components of HMS 22 are arranged according to multiple logical layers that implement the techniques of this disclosure. Each layer may be implemented by one or more modules comprised of hardware, software, or a combination of hardware and software.

Computing devices, such as computing device(s) 12, IoT devices 30, and computing devices 38 operate as clients that communicate with HMS 22 via interface layer 300. The computing devices typically execute client software applications, such as desktop application, mobile application, and web applications. Interface layer 300 represents a set of application programming interfaces (API) or protocol interfaces presented and supported by HMS 22 for the client software applications. Interface layer 300 may be implemented with one or more web servers.

As shown in FIG. 5, HMS 22 also includes an application layer 302 that represents a collection of services 310 for implementing the functionality ascribed to HMS 22 herein. Application layer 302 receives information from client applications, e.g., data from a computing device 12 or IoT device 30 (some or all of which may have been retrieved from IMD 10), and further processes the information according to one or more of the services 310 to respond to the information. Application layer 302 may be implemented as one or more discrete software services 310 executing on one or more application servers, e.g., physical or virtual machines. That is, the application servers provide runtime environments for execution of services 310. In some examples, the functionality interface layer 300 as described above and the functionality of application layer 302 may be implemented at the same server. Services 310 may communicate via a logical service bus 312. Service bus 312 generally represents a logical interconnections or set of interfaces that allows different services 310 to send messages to other services, such as by a publish/subscription communication model.

Data layer 304 of HMS 22 provides persistence for information in HMS 22 using one or more data repositories 320. A data repository 320, generally, may be any data structure or software that stores and/or manages data. Examples of data repositories 320 include but are not limited to relational databases, multi-dimensional databases, maps, and hash tables, to name only a few examples.

As shown in FIG. 5, each of services 330-334 is implemented in a modular form within HMS 22. Although shown as separate modules for each service, in some examples the functionality of two or more services may be combined into a single module or component. Each of services 330-334 may be implemented in software, hardware, or a combination of hardware and software. Moreover, services 330-334 may be implemented as standalone devices, separate virtual machines or containers, processes, threads or software instructions generally for execution on one or more physical processors. Record management service 334 may store received patient data as parameter data 350 and AF episode data 352.

Recurrence analyzer 330 may determine a likelihood of AF recurrence after an AF treatment procedure based on AF episode data 352, and in some cases other parameter data 350, generated by IMD 10 based on AF episodes detected during an observation time period prior to deciding whether to perform the procedure for patient 4. Recurrence analyzer 330 may determine the likelihood of recurrence based on application of the data as inputs one or more model(s) 354, which may include one or more probability models, machine learning models, algorithms, decision trees, and/or thresholds. In examples in which models 354 include one or more machine learning models, recurrence analyzer 330 may apply feature vectors derived from the data to the model(s).

Model(s) 354 may be developed by model configuration service 332 based on machine learning. Example machine learning techniques that may be employed to generate model(s) 350 can include various learning styles, such as supervised learning, unsupervised learning, and semi-supervised learning. Example types of algorithms include Bayesian algorithms, Markov models, Hawkes processes, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

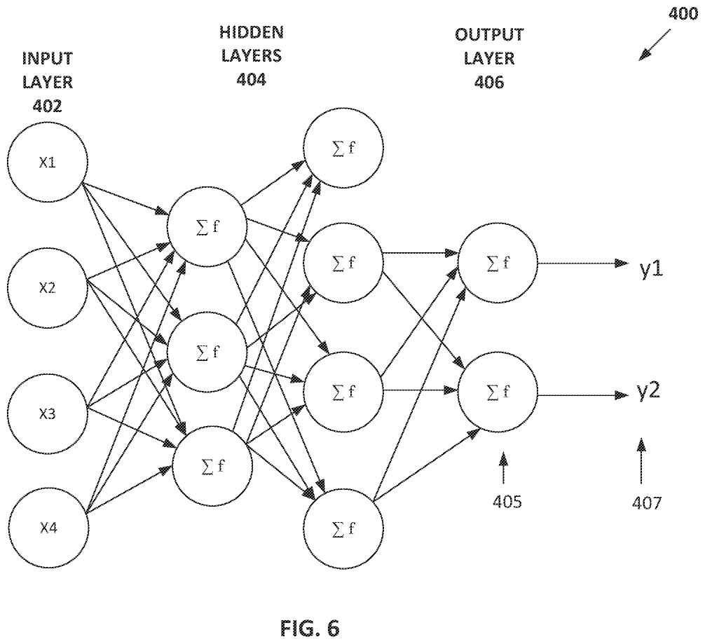

FIG. 6 is a conceptual diagram illustrating an example machine learning model 400 configured to determine a likelihood of recurrence of AF. Machine learning model 400 is an example of a deep learning model, or deep learning algorithm. One or more of IMD 10, computing devices 12, or computing system 20 (e.g., model configuration component 272 and/or 332) may train, store, and/or utilize machine learning model 400, but other devices may apply inputs associated with a particular patient to machine learning model 400 in other examples. Some non-limiting examples of machine learning techniques include Bayesian probability models, Hawkes processes, Support Vector Machines, K-Nearest Neighbor algorithms, and Multi-layer Perceptron.

As shown in the example of FIG. 6, machine learning model 400 may include three layers. These three layers include input layer 402, hidden layer 404, and output layer 406. Output layer 406 comprises the output from the transfer function 405 of output layer 406. Input layer 402 represents each of the input values X1 through X4 provided to machine learning model 400. The number of inputs may be less than or greater than 4, including much greater than 4, e.g., hundreds or thousands. In some examples, the input values may be parameters determined based on AF episode data 184, 292, 352, including those described herein, and in some cases other parameter data 182, 290, 350. In some examples, the input values may include those described in U.S. Provisional Patent Application Ser. No. 63/365,188, filed May 23, 2022 and titled “SYSTEM USING HEART RATE VARIABILITY FEATURES FOR PREDICTION OF MEDICAL PROCEDURE EFFICACY”, the entire content of which is incorporated herein by reference.

Each of the input values for each node in the input layer 402 is provided to each node of hidden layer 404. In the example of FIG. 6, hidden layers 404 include two layers, one layer having four nodes and the other layer having three nodes, but fewer or greater number of nodes may be used in other examples. Each input from input layer 402 is multiplied by a weight and then summed at each node of hidden layers 404. During training of machine learning model 400, the weights for each input are adjusted to establish the relationship between input physiological parameter values and one or more output values indicative of a health state of the patient. In some examples, one hidden layer may be incorporated into machine learning model 400, or three or more hidden layers may be incorporated into machine learning model 400, where each layer includes the same or different number of nodes.

The result of each node within hidden layers 404 is applied to the transfer function of output layer 406. The transfer function may be liner or non-linear, depending on the number of layers within machine learning model 400. Example non-linear transfer functions may be a sigmoid function or a rectifier function. The output 407 of the transfer function may be a value or values indicative of a likelihood (e.g., a probability) of recurrence of AF after PVI or another procedure to treat AF. By applying the patient parameter data to a machine learning model, such as machine learning model 400, processing circuitry of system 2 is able to determine the likelihood of AF recurrence with great accuracy, specificity, and sensitivity.

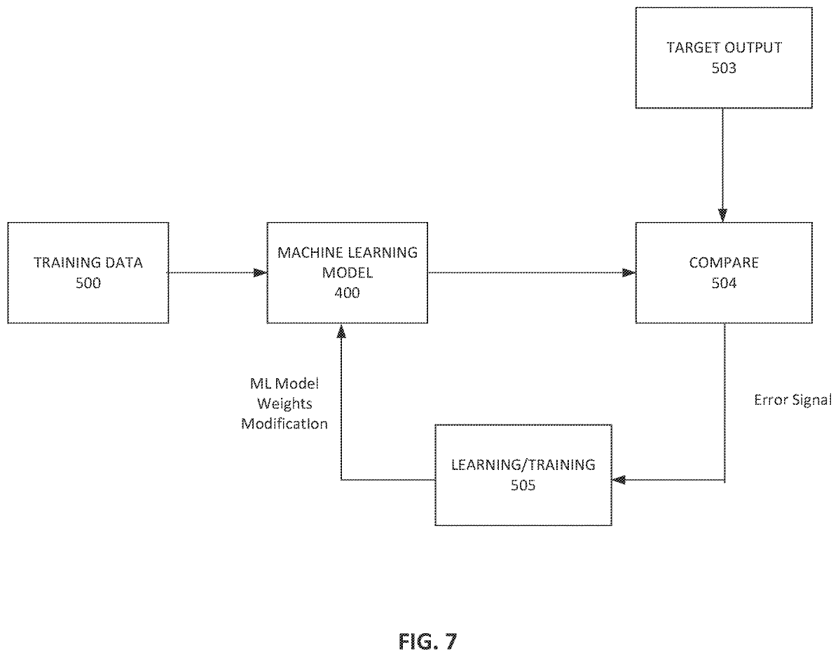

FIG. 7 is an example of a machine learning model 400 being trained using supervised and/or reinforcement learning techniques. Machine learning model 400 may be implemented using any number of models for supervised and/or reinforcement learning, such as but not limited to, an artificial neural network, a decision tree, naïve Bayes network, support vector machine, or k-nearest neighbor model, to name only a few examples. In some examples, processing circuitry one or more of IMD 10, computing device 12, and/or computing system 20 (e.g., model configuration component 272 and/or 332) initially trains the machine learning model 400 based on training set data 500 including numerous instances of input data, e.g., parameters determined from AF episode data, corresponding to and labeled as either recurrence or non-recurrence of AF. An output of the machine learning model 400 may be compared 504 to the target output 503, e.g., as determined based on the label. Based on an error signal representing the comparison, the processing circuitry implementing a learning/training function 505 may send or apply a modification to weights of machine learning model 400 or otherwise modify/update the machine learning model 400. For example, one or more of IMD 10, computing device 12, and/or computing system 20 may, for each training instance in the training set 500, modify machine learning model 400 to change a probability or other likelihood output generated by the machine learning model 400 in response to data applied to the machine learning model 400.

In various aspects, processing circuitry determines a likelihood of AF recurrence based on modeling AF episode timing as a bivariate Hawkes process. The bivariate Hawkes process is a statistical approach. The bivariate Hawkes process characterizes AF episode patterns where the AF episodes are assumed to be history dependent. The bivariate Hawkes process assumes that the heart switches between an AF state and an SR state, although the heart may take other non-AF rhythms.

The AF episode pattern is modeled by two alternating point processes {N₁(t), N₂(t), t>0}, which describe the number of transitions that have occurred up to time t: one accounting for transitions from SR to AF occurring at times (points) t_{1,1}, t_{1,2}, . . . , and another for transitions from AF to SR occurring at times t_{2,1}, t_{2,2}, . . . . Here, the first subscript denotes the type of transition (SR-to-AF is denoted 1, while AF-to-SR is denoted 2), and the second subscript denotes the transition number.

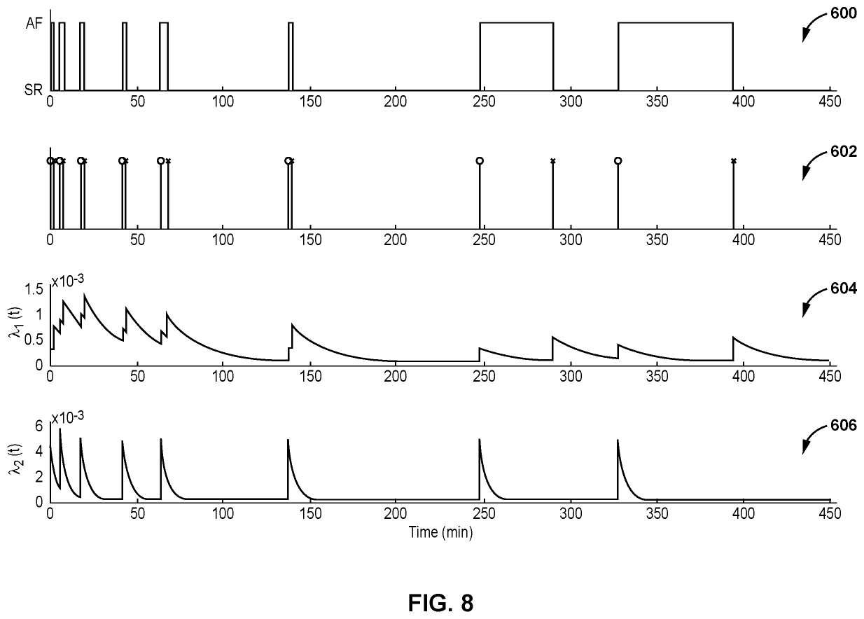

FIG. 8 is a set of charts showing heart rhythm conditions over time, heart rhythm condition transition times, a first conditional intensity function, and a second conditional intensity function according to some aspects of this disclosure. In FIG. 8, chart 600 shows an example of an AF episode pattern, with the heart rhythm condition being classified as either AF or SR over time. Chart 602 shows the transition times for the episode pattern in chart 600. The marks “o” and “x” indicate SR-to-AF and AF-to-SR transitions, respectively.

The counting processes N₁(t) and N₂(t) are defined by the following two conditional intensity functions λ₁ and λ₂:

$${λ}_{1} ( t ) = {μ}_{1} + {\sum }_{n = 1}^{2} {\sum }_{\{ k : t > {t}_{n , k} \}} {α}_{1 , n} {e}^{- {β}_{1 , n} ( t - {t}_{n , k} )}$$

$${λ}_{2} ( t ) = {μ}_{2} + {\sum }_{n = 1}^{2} {\sum }_{\{ k : t > {t}_{n , k} \}} {α}_{2 , n} {e}^{- {β}_{2 , n} ( t - {t}_{n , k} )}$$

where base intensity μₘ>0, excitation parameter α_{m,n}≥0, and decay parameter β_{m,n}≥0 for m, n=1, 2.

A characteristic of the model, exhibited in chart 604, is that the conditional intensity function λ₁(t) increases by an amount corresponding to excitation parameter α_{1,1} immediately after an SR-to-AF transition and then decreases exponentially, according to decay parameter β_{1,1}. Similarly, as illustrated in chart 606, the conditional intensity function λ₂(t) increases by an amount corresponding to excitation parameter α_{2,2} immediately after an AF-to-SR transition and then decreases exponentially, according to decay parameter β_{2,2}.

As the probability of additional transitions increases immediately after a transition, the process can account for clustering behavior. In addition to the self-excitation, both conditional intensity functions λ₁(t) and λ₂(t) contain additional terms, defined by α_{1,2} and β_{1,2} in the case of λ₁(t) and by α_{2,1} and β_{2,1} in the case of λ₂(t), which lets the counting processes influence each other (cross-excitation).

The bivariate Hawkes process in its original formulation does not impose alternating transitions, i.e., an SR-to-AF transition is not necessarily followed by an AF-to-SR transition. This situation is addressed by multiplying λ₁(t) and λ₂(t) with a binary “occurrence” function oₘ(t), where:

$${o}_{1} ( t ) = \{ \begin{matrix}1 , & {N}_{1} ( t ) = {N}_{2} ( t - {d}_{2} ) , \\ 0 , & otherwise ,\end{matrix} and$$

$${o}_{2} ( t ) = \{ \begin{matrix}1 , & {N}_{2} ( t ) \neq {N}_{1} ( t - {d}_{1} ) , \\ 0 , & otherwise ,\end{matrix}$$

which ensures that AF occurs after SR, and that SR occurs after AF, respectively. The parameters d₁ and d₂ define the minimum duration of AF and SR, respectively.

Thus, the conditional intensity functions for the alternating, bivariate Hawkes process are given by:

\tilde{λ}₁(t)=λ₁(t)o₁(t),

\tilde{λ}₂(t)=λ₂(t)o₂(t).

The structure of \tilde{λ}ₘ(t) is identical to that of the bivariate Hawkes process λₘ(t), except that a SR-to-AF transition can, once a certain time d₁ has elapsed, only be followed by an AF-to-SR transition, and so on.

FIG. 8 further shows an example of the two conditional intensity functions. Charts 604 and 606 show the conditional intensity functions (λ₁(t) for SR-to-AF transitions and λ₂(t) for AF-to-SR transitions). For reasons of clarity, λ₁(t) and λ₂(t) are displayed rather than \tilde{λ}₁(t) and \tilde{λ}₂(t).

The base intensity ratio μ is defined as:

$$μ = \frac{{μ}_{1}}{{μ}_{2}}$$

and provides information on the dominating rhythm of the analyzed segment: μ>1 indicates dominance of AF, and μ<1 indicates dominance of SR. In some implementations, the natural logarithm of the base intensity ratio μ (log(μ)) may be used instead of the base intensity ratio μ, since the logarithmic parameter exhibits a more linear behavior. The base intensity ratio μ indicates a degree of dominance of AF during the measured interval.

The decay parameter β₁, empirically given a range between 0 and 0.3, describes the degree of episode clustering, where a value of β₁ closer to 0.3 reflects fewer clusters, and a value closer to 0 reflects more clustering. Clustering may also be referred to as temporal aggregation of AF episodes.

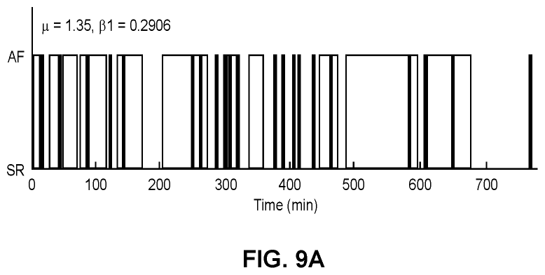

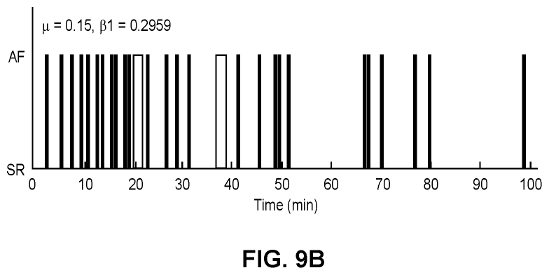

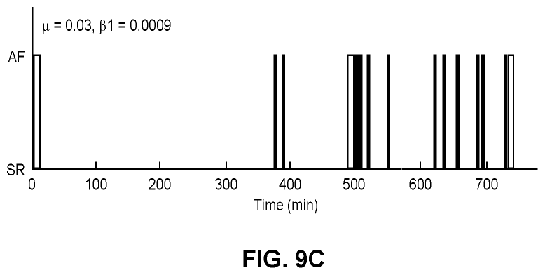

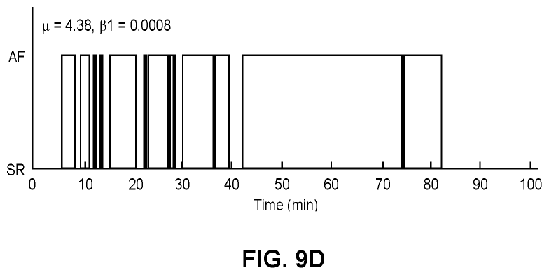

FIGS. 9A-9D are charts showing varying degrees of prevalence over time of AF episodes and clustering of AF episodes indicated using the bivariate Hawkes process according to some aspects of this disclosure. For example, the chart of FIG. 9A exhibits a high base intensity ratio (μ>1) and a low degree of clustering (β₁ near 0.3). The chart of FIG. 9B exhibits a low base intensity ratio (μ<1) and a low degree of clustering (β₁ near 0.3). The chart of FIG. 9C exhibits a low base intensity ratio (μ<1) and a high degree of clustering (β₁ near 0). The chart of FIG. 9D exhibits a high base intensity ratio (μ>1) and a high degree of clustering (β₁ near 0). In examples according to this disclosure, β₂ is not considered for prediction, as β₁ is the parameter indicative of AF episode clustering.

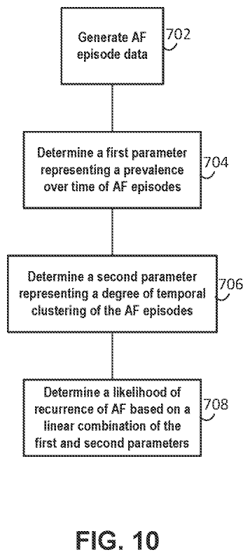

FIG. 10 is a flow chart illustrating an example process for making a pre-ablation risk assessment including determining a likelihood of post-ablation recurrence of atrial fibrillation (AF). In some examples, the process of FIG. 10 may be carried out by a medical system 2 that includes an IMD, such as IMD 10.

According to the example process of FIG. 10, IMD 10 generates AF episode data (702). For example, IMD 10 may detect AF episodes based on an ECG or other cardiac signal sensed by the IMD during a monitoring period of time. The monitoring period of time may begin a certain amount of time after implantation of IMD 10, e.g., to allow the cardiac signal to become more reliable. The monitoring period of time may include any amount of time. The monitoring period of time may include ten or more AF episodes. AF episode data may include data indicate start times of AF episodes (or SR to AF transitions), stop times of AF episodes (or AF to SR transitions), and/or AF episode durations.

Processing circuitry, e.g., processing circuitry 230 of computing device 12 and/or processing circuitry 19 of computing system 20, may determine a first parameter representing a prevalence over time of AF episodes (704). For example, to determine the first parameter the processing circuitry may determine a base intensity ratio μ providing information on the dominating rhythm of the analyzed recording. In some examples, the first parameter is a measure of or indicative of AF burden.

The processing circuitry may determine a second parameter representing a degree of temporal clustering of the AF episodes in the recording (706). For example, the processing circuitry may determine a degree of clustering 81 providing information on the grouping or clustering of AF episodes in the recording over time. In some examples, the processing circuitry determines the first parameter and the second parameter at the same time, e.g., based on application of AF episode data to a common one or more models.

The processing circuitry may determine a likelihood of recurrence of AF based on first parameter and the second parameter (708). For example, the processing circuitry may determine the likelihood of recurrence based on a linear combination of the first and second parameters:

AFRecurrenceᵣᵢₛₖ=w_{μ}*log(μ)+w_{β}_{₁}*β₁
where w_{μ} is the weight of the logarithm of the base intensity ratio μ and w_{β}_{₁} is the weight of degree of clustering β₁.

The likelihood of recurrence may be higher when the first parameter is higher and when the second parameter is lower, e.g., since likelihood of recurrence is higher when the degree of clustering is higher and the second parameter is lower when the degree of clustering is higher. To determine the likelihood of recurrence of AF in some examples, the processing circuitry may apply a model, e.g., a machine learning model, to the first parameter and the second parameter. In some examples, the processing circuitry applies a model, e.g., a Hawkes process or other probability model, to the AF episode data to determine one or both of the first parameter or the second parameter, e.g., simultaneously.

The likelihood of AF recurrence may be, as examples, a classification of responder/non-responder, low/medium/high likelihood, or a probability (e.g., from 0 to 1) or other numeric value. In some examples, the processing circuitry applies one or more thresholds to an output of a linear combination or model to determine a classification. The processing circuitry transmits information indicative of the likelihood of AF to a computing device 38 of a clinician 40 for a determination of whether to undertake the ablation or other treatment for patient 4.

Accordingly, although example systems and techniques have been shown and described, it is to be understood that all the terms used herein are descriptive rather than limiting, and that many changes, modifications, and substitutions may be made by one having ordinary skill in the art without departing from the spirit and scope of the invention. The following examples are examples of systems, devices, and methods described herein.

Example 1. A medical system comprising: an implantable medical device configured to: sense a cardiac signal of a patient; detect a plurality of episodes of atrial fibrillation (AF) over a time period based on the cardiac signal; and store AF episode data for the plurality of episodes of AF detected over the time period; and processing circuitry configured to: determine, based on the AF episode data, a first parameter representing a prevalence over the time of AF episodes and a second parameter representing a degree of temporal clustering of the AF episodes; determine a likelihood of recurrence of AF after an AF treatment procedure based on the first parameter and the second parameter; and transmit the likelihood to a computing device for presentation to a clinician.

Example 2. The medical system of example 1, wherein the processing circuitry is configured to determine the likelihood of recurrence of AF based on a linear combination of the first parameter and the second parameter.

Example 3. The medical system of example 1 or 2, wherein the likelihood of recurrence of AF is higher when the first parameter representing the prevalence over time of AF episodes is higher.

Example 4. The medical system of any one or more of examples 1 to 3, wherein the likelihood of recurrence of AF is higher when the second parameter representing the degree of temporal clustering of the AF episodes is higher.

Example 5. The medical system of any one or more of examples 1 to 4, wherein to determine the likelihood of recurrence of AF the processing circuitry is configured to apply a model to the first parameter and the second parameter.

Example 6. The medical system of example 5, wherein the model comprises a machine learning model.

Example 7. The medical system of example 6, wherein the model comprises a first model, wherein to determine the second parameter, the processing circuitry is configured to apply a second model to the AF episode data.

Example 8. The medical system of example 7, wherein the second model comprises a probability model.

Example 9. The medical system of any one or more of examples 1 to 8, wherein the degree of temporal clustering is characterized by the second parameter [β₁] representing a decay rate of a conditional intensity function λ₁(t): λ₁(t)=μ₁+

$${\sum }_{n = 1}^{2} {\sum }_{\{ k : t > {t}_{n , k} \}} {α}_{1 , n} {e}^{- {β}_{1 , n} ( t - {t}_{n , k} )} ,$$

wherein μ₁>0 represents a mean rate of sinus rhythm (SR)-to-AF transitions, wherein α₁≥0 represents an excitation parameter, and wherein t represents time.

Example 10. The medical system of any one or more of examples 1 to 9, wherein the prevalence over time of AF episodes is characterized by the first parameter [μ], wherein:

$$μ = \frac{{μ}_{1}}{{μ}_{2}} ,$$

wherein μ₁>0 represents a mean rate of sinus rhythm (SR)-to-AF transitions, and wherein μ₂>0 represents a mean rate of AF-to-SR transitions.

Example 11. The medical system of any one or more of examples 1 to 9, wherein the first parameter comprises an AF burden parameter.

Example 12. The medical system of any one or more of examples 1 to 11, wherein the AF treatment procedure comprises an ablation procedure.

Example 13. The medical system of example 12, wherein the ablation procedure comprises a pulmonary vein isolation procedure.

Example 14. The medical system of any of examples 1 to 13, wherein the implantable medical device comprises an insertable cardiac monitor comprising: a housing configured for subcutaneous implantation in the patient, the housing having a length between 40 millimeters (mm) and 60 mm between a first end and a second end, a width less than the length, and a depth less than the width; a first electrode at or proximate to the first end; and a second electrode at or proximate to the second end, wherein the insertable cardiac monitor is configured to sense the cardiac signal via the first electrode and the second electrode.

Example 15. The medical system of any one or more of examples 1 to 14, further comprising one or more computing devices comprising the processing circuitry and configured to communicate with the implantable medical device.

Example 16. A method of operating a medical system comprising an implantable medical device configured to sense a cardiac signal of a patient and detect a plurality of episodes of atrial fibrillation (AF) over a time period based on the cardiac signal, the method comprising: determining, by processing circuitry of the medical system and based on AF episode data stored by the implantable medical device for the plurality of episodes of AF detected over the time period, a first parameter representing a prevalence over the time of AF episodes and a second parameter representing a degree of temporal clustering of the AF episodes; determining, by the processing circuitry, a likelihood of recurrence of AF after an AF treatment procedure based on the first parameter and the second parameter; and transmitting, by the processing circuitry, the likelihood to a computing device for presentation to a clinician.

Example 17. The method of example 16, wherein determining the likelihood of recurrence of AF comprises determining the likelihood of recurrence of AF based on a linear combination of the first parameter and the second parameter, wherein the likelihood of recurrence of AF is higher when the first parameter representing the prevalence over time of AF episodes is higher, and wherein the likelihood of recurrence of AF is higher when the second parameter representing the degree of temporal clustering of the AF episodes is higher.

Example 18. The method of example 16 or 17, wherein the degree of temporal clustering is characterized by the second parameter [β₁] representing a decay rate of a conditional intensity function λ₁(t):

$${λ}_{1} ( t ) = {μ}_{1} + {\sum }_{n = 1}^{2} {\sum }_{\{ k : t > {t}_{n , k} \}} {α}_{1 , n} {e}^{- {β}_{1 , n} ( t - {t}_{n , k} )} ,$$

wherein μ₁>0 represents a mean rate of sinus rhythm (SR)-to-AF transitions, wherein α₁≥0 represents an excitation parameter, and wherein t represents time.

Example 19. The method of any one or more of examples 16 to 18, wherein the prevalence over time of AF episodes is characterized by the first parameter [μ], wherein:

$$μ = \frac{{μ}_{1}}{{μ}_{2}} ,$$

wherein μ₁>0 represents a mean rate of sinus rhythm (SR)-to-AF transitions, and wherein μ₂>0 represents a mean rate of AF-to-SR transitions.

Example 20. A non-transitory computer-readable storage medium comprising program instructions that, when executed by processing circuitry of a medical system comprising an implantable medical device configured to sense a cardiac signal of a patient and detect a plurality of episodes of atrial fibrillation (AF) over a time period based on the cardiac signal, cause the processing circuitry to: determine, based on AF episode data stored by the implantable medical device for the plurality of episodes of AF detected over the time period, a first parameter representing a prevalence over the time of AF episodes and a second parameter representing a degree of temporal clustering of the AF episodes; determine a likelihood of recurrence of AF after an AF treatment procedure based on the first parameter and the second parameter; and transmit the likelihood to a computing device for presentation to a clinician.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1: A medical system comprising:
an implantable medical device configured to:
sense a cardiac signal of a patient;
detect a plurality of episodes of atrial fibrillation (AF) over a time period based on the cardiac signal; and
store AF episode data for the plurality of episodes of AF detected over the time period; and
processing circuitry configured to:
determine, based on the AF episode data, a first parameter representing a prevalence over the time period of AF episodes and a second parameter representing a degree of temporal clustering of the AF episodes;
determine a likelihood of recurrence of AF after an AF treatment procedure based on the first parameter and the second parameter; and
transmit the likelihood to a computing device for presentation to a clinician.

2: The medical system of claim 1, wherein the processing circuitry is configured to determine the likelihood of recurrence of AF based on a linear combination of the first parameter and the second parameter.

3: The medical system of claim 1, wherein the likelihood of recurrence of AF is higher when the first parameter representing the prevalence over the time period of AF episodes is higher.

4: The medical system of claim 1, wherein the likelihood of recurrence of AF is higher when the second parameter representing the degree of temporal clustering of the AF episodes is lower.

5: The medical system of claim 1, wherein to determine the likelihood of recurrence of AF the processing circuitry is configured to apply a model to the first parameter and the second parameter.

6: The medical system of claim 5, wherein the model comprises a machine learning model.

7: The medical system of claim 6, wherein the model comprises a first model, wherein to determine the second parameter, the processing circuitry is configured to apply a second model to the AF episode data.

8: The medical system of claim 7, wherein the second model comprises a probability model.

9: The medical system of claim 1, wherein the degree of temporal clustering is characterized by the second parameter [β₁] representing a decay rate of a conditional intensity function λ₁(t):
$${λ}_{1} ( t ) = {μ}_{1} + {\sum }_{n = 1}^{2} {\sum }_{\{ k : t > {t}_{n , k} \}} {α}_{1 , n} {e}^{- {β}_{1 , n} ( t - {t}_{n , k} )} ,$$
wherein μ₁>0 represents a mean rate of sinus rhythm (SR)-to-AF transitions,
wherein α₁≥0 represents an excitation parameter, and
wherein t represents time.

10: The medical system of claim 1, wherein the prevalence over time of AF episodes is characterized by the first parameter [μ], wherein:
$$μ = \frac{{μ}_{1}}{{μ}_{2}} ,$$
wherein μ₁>0 represents a mean rate of sinus rhythm (SR)-to-AF transitions, and
wherein μ₂>0 represents a mean rate of AF-to-SR transitions.

11: The medical system of claim 1, wherein the first parameter comprises an AF burden parameter.

12: The medical system of claim 1, wherein the AF treatment procedure comprises an ablation procedure.

13: The medical system of claim 12, wherein the ablation procedure comprises a pulmonary vein isolation procedure.

14: The medical system of claim 1, wherein the implantable medical device comprises an insertable cardiac monitor comprising:
a housing configured for subcutaneous implantation in the patient, the housing having a length between 40 millimeters (mm) and 60 mm between a first end and a second end, a width less than the length, and a depth less than the width;
a first electrode at or proximate to the first end; and
a second electrode at or proximate to the second end,
wherein the insertable cardiac monitor is configured to sense the cardiac signal via the first electrode and the second electrode.

15: The medical system of claim 1, further comprising one or more computing devices comprising the processing circuitry and configured to communicate with the implantable medical device.

16: A method of operating a medical system comprising an implantable medical device configured to sense a cardiac signal of a patient and detect a plurality of episodes of atrial fibrillation (AF) over a time period based on the cardiac signal, the method comprising:
determining, by processing circuitry of the medical system and based on AF episode data stored by the implantable medical device for the plurality of episodes of AF detected over the time period, a first parameter representing a prevalence over the time of AF episodes and a second parameter representing a degree of temporal clustering of the AF episodes;
determining, by the processing circuitry, a likelihood of recurrence of AF after an AF treatment procedure based on the first parameter and the second parameter; and
transmitting, by the processing circuitry, the likelihood to a computing device for presentation to a clinician.

17: The method of claim 16,
wherein determining the likelihood of recurrence of AF comprises determining the likelihood of recurrence of AF based on a linear combination of the first parameter and the second parameter,
wherein the likelihood of recurrence of AF is higher when the first parameter representing the prevalence over time of AF episodes is higher, and wherein the likelihood of recurrence of AF is higher when the second parameter representing the degree of temporal clustering of the AF episodes is lower.

18: The method of claim 16, wherein the degree of temporal clustering is characterized by the second parameter [β₁] representing a decay rate of a conditional intensity function λ₁(t):
$${λ}_{1} ( t ) = {μ}_{1} + {\sum }_{n = 1}^{2} {\sum }_{\{ k : t > {t}_{n , k} \}} {α}_{1 , n} {e}^{- {β}_{1 , n} ( t - {t}_{n , k} )} ,$$
wherein μ₁>0 represents a mean rate of sinus rhythm (SR)-to-AF transitions,
wherein α₁≥0 represents an excitation parameter, and
wherein t represents time.

19: The method of claim 16, wherein the prevalence over time of AF episodes is characterized by the first parameter [μ], wherein:
$$μ = \frac{{μ}_{1}}{{μ}_{2}} ,$$
wherein μ₁>0 represents a mean rate of sinus rhythm (SR)-to-AF transitions, and
wherein μ₂>0 represents a mean rate of AF-to-SR transitions.

20: A non-transitory computer-readable storage medium comprising program instructions that, when executed by processing circuitry of a medical system comprising an implantable medical device configured to sense a cardiac signal of a patient and detect a plurality of episodes of atrial fibrillation (AF) over a time period based on the cardiac signal, cause the processing circuitry to:
determine, based on AF episode data stored by the implantable medical device for the plurality of episodes of AF detected over the time period, a first parameter representing a prevalence over the time of AF episodes and a second parameter representing a degree of temporal clustering of the AF episodes;
determine a likelihood of recurrence of AF after an AF treatment procedure based on the first parameter and the second parameter; and
transmit the likelihood to a computing device for presentation to a clinician.
